(19) United States
(12) Patent Application Publication
(10) Pub. No.: US 2014/0296830 A1
(43) Pub. Date: Oct. 2, 2014

(54) METHODS OF ADMINISTERING DRUGS IN AN IMPLANTABLE MULTI-CHAMBER PUMP

(71) Applicant: Jazz Pharmaceuticals, Inc., Palo Alto, CA (US)

(72) Inventors: Susan P. Gibson, Menlo Park, CA (US); Geertrui F. Vanhove, Menlo Park, CA (US)

(73) Assignee: Jazz Pharmaceuticals, Inc., Palo Alto, CA (US)

(21) Appl. No.: 14/228,192

(22) Filed: Mar. 27, 2014

Related U.S. Application Data
(60) Provisional application No. 61/806,084, filed on Mar. 28, 2013.

(51) Int. Cl. A61M 5/14; A61M 5/142

(52) U.S. Cl. CPC A61M 5/1408; A61M 5/14276; A61M 2005/14208
USPC 604/512

(57) ABSTRACT

One embodiment of the present invention is a method for reducing pain using a multi chamber pump to separately administer multiple drugs. For example, one chamber may contain an omega conopeptide, such as ziconotide, and the other chamber or chambers may contain one or more other drugs, which may include of morphine, hydromorphone, fentanyl, sufentanil, bupivacaine, baclofen, clonidine, and buprenorphine, or their pharmaceutically acceptable salts thereof. Other applications of the present invention include methods for treating severe chronic pain due to cancer, failed back syndrome, CRPS, neuropathic pain, mixed neuropathic and nociceptive pain.

The present application claims the benefit of U.S. Provisional Application 61/806,084, filed Mar. 28, 2013, which is incorporated herein by reference in its entirety.

### TECHNICAL FIELD

The present invention relates to a method for reducing pain using a multi chamber pump to separately administer multiple drugs. For example, one chamber may contain an omega conopeptide, such as ziconotide, and the other chamber or chambers may contain one or more other drugs such as morphine, hydromorphone, fentanyl, sufentanil, bupivacaine, baclofen, clonidine, and buprenorphine, or their pharmaceutically acceptable salts thereof.

### BACKGROUND

Omega conopeptides have been provided for analgesia alone and in combination with other analgesics. This administration can be by an implantable pump. It has been found that the admixture of omega conopeptides with other compounds can lower the stability of omega conopeptides and can cause corrosion in some pumps.

It has been shown that ziconotide stability is affected by the addition or admixture of other compounds. See U.S. Pat. No. 7,268,109 and various publications from Shields, such as:

Chemical Stability of Admixtures Combining Ziconotide With Baclofen During Simulated Intrathecal Administration, Neuromodulation: Technology at the Neural Interface, Vol. 10, Supplement 1, 2007; Chemical Stability of Ziconotide-Clonidine Hydrochloride Admixtures with and Without Morphine Sulfate During Simulated Intrathecal Administration, Neuromodulation: Technology at the Neural Interface, Vol. 10, Supplement 1, 2007; Chemical

Stability of Admixtures Combining Ziconotide with Fentanyl or Sufentanil During Simulated Intrathecal Administration, International Jour. Pharm. Compounding, Vol. 12, No. 5, Sept/Oct 2008; Chemical Stability of Admixtures Containing Ziconotide 25 mcg/mL and Morphine Sulfate 10 mg/mL or 20 mg/mL During Simulated Intrathecal Administration, International Jour. Pharm. Compounding, Vol. 12, No. 6, Nov/Dec 2008; Vol. 36, No. 1 July 2008, Letters; Chemical Stability of Admixtures Combining Ziconotide with Morphine or Hydromorphone During Simulated Intrathecal Administration, 2005 Int'l Neuromodulation Society, Neuromodulation, Vol. 8, No. 4, 2005, p 257-263; and Chemical Stability of an Admixture Combining Ziconotide and Bupivacaine During Simulated Intrathecal Administration, Neuromodulation: Technology at the Neural Interface, Vol. 10, Supplement 1, 2007. All of the above references are hereby incorporated by reference in their entireties. The above articles show combinations of various drugs including ziconotide with morphine, hydromorphone, and baclofen.

For example, the Shields work shows that ziconotide was less stable when mixed with morphine sulfate or hydromorphone. For example, Table 2 of U.S. Pat. No. 7,268,109 shows that ziconotide was less stable when combined with these two agents. Stability above 80% was achieved for less time as the concentration of the other compound increased. Table 3 of U.S. Pat. No. 7,268,109 also shows that ziconotide was less stable when combined with these two agents. Shields et al, Chemical Stability of Admixtures Combining Ziconotide With Baclofen During Simulated Intrathecal Administration, Neuromodulation: Technology at the Neural Interface, Vol. 10, Supplement 1, 2007 also show that baclofen negatively affects ziconotide stability.

Implantable pumps have been known and widely utilized for many years. Typically, pumps of this type are implanted into patients who require the delivery of active substances or medication fluids to specific areas of their body. For example, patients that are experiencing severe pain may require painkillers daily or multiple times per day. Absent the use of an implantable pump or the like, a patient of this type would be subjected to one or more painful injections of such medication fluids. In the case of pain associated with more remote areas of the body, such as the spine, these injections may be extremely difficult to administer and particularly painful for the patient. Furthermore, attempting to treat conditions such as this through oral or intravascular administration of medication often requires higher doses of medication and may cause severe side effects. Therefore, it is widely recognized that utilizing an implantable pump may be beneficial to both a patient and the treating physician.

### SUMMARY OF THE INVENTION

One embodiment of the invention is a method for reducing pain in a patient, comprising: intrathecally administering to a patient in need thereof (a) a pharmaceutical formulation comprising an effective amount of an omega conopeptide and an anti-oxidant, and (b) an effective amount of one or more compounds selected from the group consisting of morphine, hydromorphone, fentanyl, sufentanil, bupivacaine, baclofen, clonidine, buprenorphine, and their pharmaceutically acceptable salts thereof, wherein the pharmaceutical formulation and the one or more compounds are administered by a drug dispensing implantable pump having a chamber for the omega conopeptide and one or more chambers for the one or more compounds to ensure that the pharmaceutical formulation retains at least 80% of the ziconotide activity at 37° C. for at least 7 days.

Another embodiment of the invention is a method for reducing pain in a patient, comprising: administering to a patient in need thereof (a) a pharmaceutical formulation comprising an effective amount of an omega-conopeptide and an anti-oxidant, and (b) an effective amount of a compound selected from the group morphine, hydromorphone, baclofen, and their pharmaceutically acceptable salts thereof, wherein said administering is by intrathecal infusion using a drug dispensing implantable pump having a chamber for the omega-conopeptide and a separate chamber for the other compound(s), so that the omega-conopeptide retains its stability in the pharmaceutical formulation.

A further embodiment of the invention is a method for reducing severe, chronic pain in a patient having an implantable pump, comprising: placing ziconotide in a first chamber of a pump implanted in a patient having severe, chronic pain, placing morphine in a second chamber of the pump, and administering the ziconotide and the morphine so that the ziconotide retains at least 80% of the ziconotide activity at 37° C. for at least 7 days.

In any of the above embodiments, an operator can employ a pump that can adjust the flow rates of the separate chambers independently of each other for independent dose adjustments or can adjust the flow rates based on the incidence of adverse events from any of the compounds. Further, the above embodiments can be used to treat severe chronic pain due to cancer, failed back syndrome, CRPS, neuropathic pain, nociceptive pain, and/or mixed neuropathic and nociceptive pain.

### DETAILED DESCRIPTION OF THE INVENTION

One embodiment of the present invention is directed to a method of combination drug therapy in which a patient is administered multiple drugs in the same infusion pump but the drugs are not in contact with each other until administration to the patient, such as into the cerebrospinal fluid (CSF). Multiple drugs can be co-administered by any of a number of routes of administration, particularly by intrathecal administration, including continuous intrathecal administration and bolus intrathecal administration. This invention fulfills a need for a method of dispensing other drugs, especially analgesic and anti-spasticity drugs in combination with an omega-conopeptide so as to enhance the stability of the omega conopeptide while preventing pump corrosion. The method also enables the management and reduction the side effects related to each drug therapy, allows flexibility in titration of each drug separately, speeds up the onset of drug action and helps avoid drug toxicity and degradation.

One embodiment of the present invention provides a method for reducing severe chronic pain in a human patient. The method comprises administering to a subject an effective amount of an omega conopeptide, preferably ziconotide, combined with one or more traditional compounds that may include analgesics, such as opioids, local anesthetics, adrenergic agonists, glutamate receptor antagonists, NMDA antagonists, and other analgesic agents.

Opioids, omega conopeptides and other drugs have been provided for analgesia alone and in combination with other intrathecal treatments to treat a variety of conditions, including but not limited to, spasticity and/or severe chronic pain due to cancer, failed back syndrome, complex regional pain syndrome (CRPS), neuropathic pain, nociceptive pain, and/or mixed neuropathic and nociceptive pain, etc. This administration can be by an implantable pump that provides continuous infusion or bolus injection. It has been found that the admixture of multiple products in a single chamber of a pump can cause corrosion in some pumps. Pump corrosion can cause motor failure and can even lead to death. Single chamber pumps also have only one flow rate, so admixtures of multiple drugs cannot easily be adjusted for the appropriate flow rate and dose of each drug. When adverse effects occur, the dose of the offending agent needs to be reduced. With single-chamber pumps, the concentration of the non-offending drug would need to be increased to make up for the reduction in flow rate of the offending drug. With single-chamber pumps, adjustments of dose of individual drugs is not possible and options are only to reduce the dose of both drugs, increase the dose of both drugs, or empty the pump (throwing away the remaining drugs) and refill the pump with a new mixture.

Opioids suitable for the present invention include morphine and all its salts thereof such as morphine sulfate and morphine gluconate, hydromorphone, fentanyl, fentanyl citrate, sufentanil, sufentanil citrate, methadone, buprenorphine, and meperidine. Local anesthetics include bupivacaine, ropivacanine, and tetracaine. Adrenergic agonists include clonidine and tizanidine. NMDA antagonists include ketamine, dextrorphan, dextromethorphan and memantine. Other analgesic and agents with other effects include adenosine, aspirin, baclofen, droperidol, gabapentin, ketorolac, midazolam, neostigmine, octreotide (a somatostatin analogue), midazolam (a sedative/hypnotic) and valproate (an anti-epileptic).

In one embodiment, an omega conopeptide such as ziconotide is placed in one chamber of a multi chamber pump and one or more of the other compounds listed above is placed in one or more other separate chambers. 1, 2, 3 or more compounds may be admixed and placed in the separate chamber. These compounds for example include morphine, hydromorphone, fentanyl, sufentanil, bupivacaine, baclofen, clonidine, and buprenorphine, or their pharmaceutically acceptable salts thereof.

The system and method of the present invention allow for varying and separating flow rates and doses, are useful to manage adverse events, allow the combination of compounds that have complementary or supplementary mechanism of action, allow for titration flexibility, may speed of onset of action, and possibly reduce opioid induced hyperalgesia. The system and method provide stability for omega conopeptides, like ziconotide, avoid toxicity and unwanted degradants, and eliminate or lower pump corrosion.

In one embodiment, the pump of the present invention is used for intrathecal administration, epidural administration, and administration to other sites in the body using an implantable pump. A preferred route of administration is intrathecal administration.

### Omega Conopeptides

Omega conopeptides, also known as omega conotoxins, are a group of small (24-29 amino acids), disulfide-rich polypeptides, found in the venoms of predatory marine snails that belong to the genes Conus. All omega-conopeptides bind to N-type voltage sensitive calcium channels (VSCC), which are found exclusively in neurons, although the binding affinities for specific VSCC subtypes may differ. In response to nerve cell membrane depolarization, N-type VSCCs open and permit calcium entry that results in neurotransmitter release. N-type VSCCs are abundant in the Rexed laminae I and II of the dorsal horn of the spinal cord, where primary afferent fibers in the pain signaling synapse for the first time. Omega conopeptides bind to N-type VSCCs in the Rexed laminae I and II and blocks calcium transport into the presynaptic terminal, thereby blocking neurotransmitter release. By blocking calcium entry at N-type VSCCs in this location, pain signals, including those that develop after peripheral nerve injury and characterize peripheral neuropathies, are less easily transmitted or are blocked completely.

A preferred omega conopeptide useful for this invention is ziconotide, which is available commercially as PRIALT®. Ziconotide (SNX-111), a 25-amino acid peptide, is a synthetic version of a naturally-occurring peptide found in the venom of the marine snail Conus magus. Ziconotide specifically and selectively binds to VSCCs.

### Omega-Conopeptides and Treatment of Chronic and Neuropathic Pain

Treatment with omega conopeptides, preferably ziconotide, is useful in preventing progression of neuropathic pain. Analgesic omega-conopeptides are effective as analgesic agents both in traditional opiate-sensitive models of nociceptive pain, such as the Rat Tail-Flick model or the rat formalin model, as well as in opiate-resistant models of pain, such as allodynia model of neuropathic pain.

Ziconotide has a unique combination of pharmacological actions. Specifically, intrathecally-administered ziconotide is more potent, longer acting, and more specific in its actions than are traditional neuropathic pain targeting drugs such as morphine or clonidine.

Ziconotide is used to treat severe chronic pain such as neuropathic pain. Ziconotide, unlike morphine, does not suppress respiratory function and does not have addiction potential. Once a therapeutic dose is reached, tolerance does not appear to develop as it does with opioids.

Ziconotide is effective in both non-neuropathic (visceral, somatic) and neuropathic cancer pain and in non-malignant neuropathic pain states. Co-administration with an opioid, such as morphine, may reduce opioid induced hyperalgesia.

### Stability of Ziconotide

Dilute solutions of omega-conopeptides are generally unstable in solution, as evidenced by oxidation of methionine residues and reduction or loss of biological activity. In particular, ziconotide, which contains a methionine at position 12, is approximately 10-fold less potent in binding to omega-conopeptide MVIIA binding sites when its methionine is oxidized and present in the sulfoxy form. Omega-conopeptides can, however, be significantly stabilized in solution by preventing oxidation of methionine residues present in the peptide structure. Ziconotide oxidation, for example, can be prevented by addition of acetate buffer to the composition. Buffers containing acetate buffer, pH 4-5, improve stability of the compound considerably. Solutions of ziconotide in which the peptide concentration is less than about 0.1 mg/ml oxidize rapidly when dissolved in water, saline, or a number of buffers used in the art of peptide chemistry. Solutions of ziconotide ranging from 0.01-0.1 mg/ml are stable at 45° C. for weeks when stabilized with acetate buffer (pH 4-5). Buffers containing 50 μg/ml methionine are effective in stabilizing ziconotide when either acetate buffer or acidified saline (pH 4-5) is used to buffer the solution.

Advantages of Combination Therapy with Omega Conopeptides and Other Analgesic Compounds

Combination therapy of omega conopeptides, such as ziconotide, with other analgesic compounds offers several advantages over single drug administrations. First, administration of a combination drug therapy may allow dose reduction of the individual drug components, which may reduce development of drug tolerance to either or both drugs, and may reduce the likelihood of drug interactions. The reduced concentration of each drug also limits the dose dependent side effects of the drugs. In addition, the effective dose (ED50) is reduced because a lower concentration of each drug may be needed to achieve therapeutic effect.

Second, the administration of a combination of two drugs, which utilize different mechanisms to interrupt intractable or chronic pain mechanisms, magnifies the beneficial effects of either or both drugs in an additive or synergistic fashion.

Third, intrathecal administration of ziconotide in combination with another drug decreases or eliminates the usual sequelae of chronic intrathecal catheterization with morphine such as granuloma formation at the catheter tip.

However, not all drugs are compatible in terms of stability and activity. Ziconotide, when stored at μg/mL concentration, is particularly subjected to interaction with other drugs in the admixture. For intrathecal infusion, the admixture of drugs is stored at 37° C. over a period of one week to a month or longer, it is important that ziconotide retains its activity over 80%, preferably over 90% during storage and administration.

### Other Compounds

### Morphine

Morphine (7,8-didehydro-4,5-epoxy-17-methylmorphinan-3,6-diol) is an opium alkaloid that has potent analgesic properties toward all types of pain. Also included are other morphine compounds such as morphine gluconate and sulfate. Morphine readily causes addiction and dependency, develops tolerance where increasing dosing still do not achieve pain relief, and exhibits withdrawal symptoms if the dose is quickly reduced or withdrawn. INFUMORPH® (morphine sulfate) is a fairly stable salt prepared by neutralizing morphine with dilute sulfuric acid. Morphine sulfate does lose water of hydration and darkens on exposure to air and light. Typical intrathecal doses of morphine alone range between 0.5 and 75 mg/day, and usually between 2 and 20 mg/day.

### Hydromorphone

DILAUDID® (Hydromorphone Hydrochloride; dihydromorphinone hydrochloride) is a synthetic derivative of morphine prepared by the catalytic hydrogenation and dehydrogenation of morphine under acidic conditions using a large excess of platinum or palladium. Hydromorphone is a substitute for morphine (5 times as potent) but has approximately equal addicting properties and a shorter duration of action. One advantage of hydromorphone over morphine is that it gives less daytime sedation or drowsiness. Typical daily intrathecal dosages of hydromorphone range between 0.05 and 15 mg/day.

### Bupivacaine

MARCAINE® (Bupivacaine hydrochloride) is a sodium channel blocker that is used clinically both as a primary local anesthetic agent and as an adjuvant spinal analgesic. Bupivacaine is a local anesthetic of the amide class similar in chemical structure and properties to meprivacaine. The duration of action of bupivacaine is 2 to 3 times longer than that of tetracaine. The potency of bupivacaine is comparable to tetracaine, but both are about 4 times that of mepivacaine and lidocaine. Typical intrathecal dosages of bupivacaine alone range between 1 and 100 mg/day, usually between 5 and 15 mg/day.

### Ropivacaine

Ropivacaine is an amide-type local anesthetic with a relative affinity for A-delta and C fibers over A-beta fibers that makes it a choice for analgesia without motor loss. Ropivacaine has less affinity for motor blockade with effective sensory blockade when compared to bupivacaine, and lower lipid solubility than buipvacaine. Compared to bupivacaine, ropivacaine is less toxic, more selective for sensory versus motor nerves between the sensory and motor blockage, and has lower solubility resulting in greater spinal segmental spread. Compared to bupivacaine, ropivacaine has a shorter duration of action and biphasic time-dependent pharmacokinetics. (G. Bennett et al. (2000), Evidence-Based Review of the Literature on Intrathecal Delivery of Pain Medication, Journal of Pain and Symptom Management, 20: S12-S11).

### Clonidine

DURACLON® (Hydromorphone; 2,6-dichloro-N-2-imidazolidinylidenebenzenamine) is an antagonist at α₂ adrenergic receptors, P₁ purinergic receptors, and H₂ histamine receptors. Clonidine is used as a central antihypertensive drug and also abolishes most symptoms of opiate withdrawal. Clonidine is used clinically to treat both acute (postoperative) pain and chronic pain syndromes. Known side effects of clonidine when used clinically for the management of pain include hypotension and bradycardia. Typical daily intrathecal dosages of clonidine alone range between 10 and 400 μg, usually between 25 and 75 μg/day.

### Baclofen

Baclofen (γ-amino-β-(p-chlorophenyl)butyric acid) is a 4-chlorophenyl derivative of γ-aminobutyric acid (GABA) that acts as a selective agonist for the GABA_{B} receptor and inhibits the release of other neurotransmitters in the central nervous system. Baclofen is used for its antispastic (muscle-relaxing) effects, and is especially indicated for intractable spasticity caused by spinal cord injury or multiple sclerosis. Baclofen also possess analgesic properties and is antinociceptive when administered parenterally or intrathecally in rats. The typical dosage range of baclofen alone for intrathecal administration is 20-2000 μg/day, and usually 300-800 μg/day.

Fentanyl Citrate SUBLIMAZE®. (Fentanyl citrate; (N-(1-phenethyl-4-piperidyl)propionanilide citrate) is an anilide derivative with analgesic activity 50 times that of morphine in man. Fentanyl Citrate is used primarily as an adjunct to anesthesia, and has a rapid onset (4 minutes) and a short duration of action. Side effects similar to those of other potent analgesics are common—in particular, respiratory depression and bradycardia. Fentanyl citrate has dependence liability. Fentanyl is typically administered intrathecally at a dose of 10 μg/hour.

### Sufentanil Citrate

Sufentanil citrate (N-[4-(methoxymethy)-1-[2-(2-thienyl)ethyl]-4-piperidinyl]-N-phenylpropan-amide 2-hydroxy-1,2,3-propanetricarboxylate) is a potent opioid analgesic. Sufentanil is typically administered intrathecally at doses ranging from 0.1 to 1.5 μg/hour.

### Concomitant Therapy

Concomitant therapy using omega-conopeptides at various concentrations in combination with any one or more of several drugs at various concentrations are envisioned, including but not limited to morphine, methadone, hydromorphone, buprenorphine, meperidine, fentanyl (e.g. fentanyl citrate), sufentanil (e.g. sufentanil citrate), bupivacaine, ropivacaine, tetracaine, clonidine, tizanidine, dextrorphan, dextromethorphan, memantine, ketamine, octreotide, valproate, baclofen, midazolam, neostigmine, aspirin, adenosine, gabapentin, pregabalin, ketorolac, octreotide, or droperidol, (or a pharmaceutically acceptable salt thereof), wherein ziconotide retains its potency and is physically and chemically compatible with the analgesic compound.

The pharmaceutical formulations of the present invention are suitable for intrathecal administration. A preferred formulation is stable in a drug dispensing implantable pump at 37° C. for at least 14, 21, 28, 35, 42, 49 60, and 90 days.

### Method for Treating Patients Including Reducing Pain

The present invention provides a method for reducing pain, potentially severe chronic pain, in a patient. The method comprises administering to a patient an effective amount of an omega-conopeptide and an effective amount of an analgesic compound, wherein the omega-conopeptide and the analgesic compound are separately administered, but otherwise compatible and retain both activities during the administration. The omega-conopeptide and the analgesic compound are each in a separate formulation and can be co-administered simultaneously or sequentially.

The pharmaceutical formulation can be administered in a variety of routes of administration, including but not limited to regional or systemic, parenteral, subcutaneous, intraperitoneal, intravascular, perineural, epidural, and most particularly, intrathecal. Intrathecal delivery of drugs can be done by a bolus injection intermittent pulse or a continuous infusion. A bolus injection is defined as the injection of a drug (or drugs, also referred to as compounds) at once. Intermittent pulse involves bolus doses delivered through the pump. Continuous infusion is defined as the administration of a drug or drug combination continuously over a prolonged period of time. It can be at a constant flow rate or variable flow rate.

The formulations may be created in a variety of ways, depending upon the manner of introduction. The concentration of each drug in the drug formulation depends upon the route of administration. Generally, dosages and routes of administration of the compounds are determined according to the site of the pain and the size of the subject, according to standard pharmaceutical practices. Drug concentration may vary to increase the dose for a given flow rate. For example, a starting dose may be increased by refilling the pump with a high concentration of an active ingredient for use at the same flow rate or a lower flow rate. Lower volumes and flow rates may be advantageous to reduce or eliminate negative side effects of intrathecal ziconotide administration, such as dizziness, mental confusion and so forth. See the Prialt® product insert. Without being bound by theory, it may be that higher volumes or flow rates drive the ziconotide up into the brain from the point of intrathecal administration. Higher concentrations and low flow rates could potentially keep ziconotide limited to the injection site.

A therapeutically effective dose is an amount effective to produce a significant reduction in a condition such as chronic or neuropathic pain. The dose levels can be estimated for new compounds, by comparison with established effective doses of known compounds with structural similarities, taking into consideration predicted variations in bioavailability, biodistribution, and other pharmacokinetic properties, as can be empirically determined by persons skilled in the art. It is contemplated that dosages of drugs used in combination drug therapy are the same or lesser concentration than the concentration of each drug when administered alone by the same route of administration.

Drugs may be started at a low level and titrated upward to achieve the proper level of pain relief. Therapeutic effect will be discussed with reference to pain relief, however, other treatments are also contemplated (like spasticity for baclofen). Also, a patient may be transitioned from a higher dose of a compound such as morphine, to a lower dose using the pump and its ability to vary the concentration or the dose downward. Transitioning to a lower dose allows “weaning” from a particular compound, such as morphine for example.

Concomitant administration can be at any time after the onset of the severe chronic pain, or before an event known to elicit conditions of chronic or neuropathic pain.

In one embodiment of the invention, the method for reducing pain comprises: intrathecally administering to a patient in need thereof (a) a pharmaceutical formulation comprising an effective amount of an omega conopeptide and an anti-oxidant, and (b) an effective amount of one or more compounds selected from the group consisting of morphine, hydromorphone, fentanyl, sufentanil, bupivacaine, baclofen, clonidine, buprenorphine, and their pharmaceutically acceptable salts thereof. A preferred omega-conopeptide is ziconotide. A preferred route of administration is intrathecal administration, including bolus intrathecal injection or continuous intrathecal infusion. The omega-conopeptide and the analgesic compound can each be in a separate formulation and be co-administered simultaneously or sequentially.

In another embodiment of the invention, the method comprises: administering to a patient in need thereof (a) a pharmaceutical formulation comprising an effective amount of an omega-conopeptide and an anti-oxidant, and (b) an effective amount of a compound selected from the group morphine, hydromorphone, baclofen, and their pharmaceutically acceptable salts thereof. A preferred omega-conopeptide is ziconotide. The omega-conopeptide and the other compound are in a separate formulation and co-administered simultaneously or sequentially. In a further embodiment, the method comprises: placing ziconotide in a first chamber of a pump implanted in a patient having severe, chronic pain, placing morphine in a second chamber of the pump, and administering the ziconotide and the morphine so that the ziconotide retains at least 80% of the ziconotide activity at 37° C. for at least 7 days.

One embodiment of the present invention is also directed to a method for reducing intrathecal opioid-induced granulomas. The method comprises intrathecally administering to the patient an omega conopeptide. The method further comprises separately administering to the patient via intrathecal infusion an effective amount of another compound selected from the group consisting of morphine, bupivacaine, clonidine, and baclofen, or their pharmaceutically acceptable salts thereof.

Intrathecal infusion can be administered via an implantable pump. There are multiple types of implantable programmable pumps that are commercially available, such as the Medtronic SYNCHROMED® infusion pump, Codman 3000, made by Johnson and Johnson, and PROMETRA®, made by Flowonix and others. Palyon™ Medical Corporation has developed a programmable Implantable Drug Delivery System (IDDS), which has on-board flow sensors to maximize safety and to ensure the drug is being delivered at the rate set by a physician; the sensors work with the pump's valve to create a closed-loop system which can self-adjust when necessary.

In one embodiment, the present invention separates the omega conopeptide and another drug for their intrathecal administration. In one embodiment, the invention uses a dual chamber pump to separately contain each compound for administration. Dual chamber pumps are one aspect of the invention as well as separate pumps for each compound.

In one embodiment of the present invention, the omega conopeptide, for example ziconotide, is placed into one chamber of a multi-chamber pump and a second compound is placed into a different chamber. Other embodiments use 3, 4, 5, 6, 7, or more chambers in the implantable pump. The non-ziconotide compounds listed above can be placed into their own separate chambers and some compounds can be admixed if the admixture does not negatively affect the two compounds.

Any multiple-chamber implantable pump known in the art are suitable to be used for the present invention. For example, the multi-reservoir implantable pumps described in U.S. Pat. Nos. 8,034,030 and 8,034,029, which are incorporated herein by reference in their entireties, are suitable to be used for the present invention. Also, the multi-reservoir dual drug delivery devices using micromachined elastic metal structures and silicon microvalves described by Evans et al (Proceedings of the IEEE International Conference on Micro Electro Mechanical Systems (MEMS), pp. 252-55 (2008)), which is incorporated herein by reference in its entirety, are also suitable to be used for the present invention

In one embodiment, the present invention includes a programmable pump to selectively administer the above compounds to the patient in a predesigned method. For example, ziconotide has side effects when delivered to the brain, such as dizziness and mental confusion. See the PRIALT® package insert. One method of the present invention involves administering smaller doses of higher concentrated solutions of ziconotide in continuous or intermittent doses (pulses). For example, 1, 2, 3, 4, 5, 6, 7, 8, 9, 10 or more pulses per day may be administered to the patient for pain relief. In another embodiment, the compounds are administered continuously to the patient. In another embodiment, the compounds are continuously administered at different flow rates throughout the day for each compound. For example, ziconotide may be independently administered continuously at a constant flow rate, at a changeable flow rate, or multiple pulses. The additional compounds may be administered in the same ways, but at different programs. For example, ziconotide can be independently administered in a continuous flow or two or more pulses per day and morphine administered at a constant flow. Other programs can be devised which incorporate the above options. The flow rate may be controlled by the patient or the health care professional.

Implantable pumps may be of the programmable type. Pumps of this type provide variable flow rates, typically through the use of a solenoid pump or a peristaltic pump. The flow can even be stopped in some embodiments. In the solenoid pump, the flow rate of medication fluid can be controlled by changing the stroke rate of the pump. In the peristaltic pump, the flow rate can be controlled by changing the roller velocity of the pump. However, both of these types of programmable pumps require intricate designs and complicated controlling mechanisms. As such, it is more desirable to utilize pumps having designs similar to the aforementioned constant flow pumps.

It is useful to have a variable flow rate pump, or at least a pump having the capability of multiple fixed flow rates. While a constant flow of medication such as a painkiller may indeed be useful in dulling chronic pain, there may be times when a patient may require additional pain relief. With a constant flow pump, the flow rate is fixed, so the physician or medical professional may only provide such relief by direct injections of painkillers and the like through the aforementioned bolus port, or by increasing the overall active substance concentration of the drug housed in the pump. While indeed useful, the former amounts to nothing more than additional injections, something the pump is designed to circumvent. In addition, the latter may be considered less convenient for the physician or medical professional, since it requires choosing a different concentration of medicine, rather than merely adjusting the flow rate of the already present medication via an external programmer, as would be done with a programmable pump.

In one embodiment of the invention, a dual chamber, implantable pump is used to contain ziconotide and an additional compound, such as morphine. Each chamber can house either the ziconotide and morphine separately to maintain stability and to guard against corrosion of pump components when the two compounds are admixed.

In one embodiment of the invention, a dual chamber, implantable pump is used to contain ziconotide and an additional compound, such as baclofen. Baclofen is used to treat spasticity. Spasticity patients also often have chronic pain, so treatments for both the spasticity and chronic pain are needed continually. The dual or multi chamber pump has separate chambers to house ziconotide separate from the baclofen to maintain ziconotide stability, reduce unwanted degradants and to guard against corrosion of pump components when the two compounds are admixed. See U.S. Pat. No. 7,268,109. One of ordinary skill in the art is familiar with dual chamber pumps, however, the pumps disclosed and claimed in U.S. Pat. No. 8,034,030 ('030) and 8,034,029 are examples.

For example, FIGS. 1 and 2 of U.S. Pat. No. 8,034,030 show an implantable pump which can be a constant flow pump including a housing, which defines an interior having three reservoirs or chambers. A chamber is formed between two flexible membranes, while another chamber is formed between a top portion of housing and membrane, and a third chamber is formed between a bottom portion of housing and membrane. It is noted that flexible membranes may be of any design known in the art, for example, a membrane like that disclosed in U.S. Pat. No. 5,814,019, the disclosure of which is hereby incorporated by reference herein. These chambers are designed and configured to separately receive and house drugs for the relief of pain, such as ziconotide and morphine, while the third chamber is designed and configured to contain a propellant which expands isobarically under constant body heat. A tool for refilling an implantable drug pump is shown in U.S. Patent Publication No. 2012/0234433 which is hereby incorporated by reference.

The pump further includes a first replenishment port formed in housing. Essentially, first replenishment port is an opening formed in both top portion and bottom portion of the housing. This port is preferably covered by a first septum, which is capable of being pierced by an injection needle and, upon removal of such needle, is capable of automatically resealing itself. Septa of this type are well known to those of ordinary skill in the art. As pump is designed to medicate a patient over a limited period of time, the first replenishment port is utilized for replenishing chamber when empty or near empty. In addition, housing preferably includes a second, replenishment port for replenishing the chamber with an active substance like ziconotide or morphine as examples, through the connection formed by second passage. Similar to first replenishment port, second replenishment port is covered by a second septum.

During a replenishment procedure, a physician and/or other medical professional typically inserts an injection needle into an area of a patient's body where the pump is located, such that it may pierce one of a first septum or second septum. Thereafter, the operation of the needle causes injection of a solution from the needle to pass into either chamber through a passage or chamber. It is noted that the particular dimension of a pump and/or the patient's need may require the process to be repeated at given intervals, for example, quarterly, monthly, weekly, etc. In addition, as will be more fully discussed below, the replenishment process may be performed so as to vary the particular flow rate or concentration of a medication fluid to the patient. The pump, as shown in FIG. 1 of '030 Patent, also includes an outlet catheter for remote delivery of a fluid contained within the chambers to a specific location within the body of a patient. In one embodiment of the invention, the catheter is intrathecally inserted at an area of the spine estimated to best reduce the pain. The catheter may be any well-known catheter suitable for directing a medication fluid or the like to a location away from the pump. For example, the catheter may direct medication fluid from a pump implanted at or near the surface of a patient's body or abdomen to the spinal or other remote area. A first and a second flow resistor can be connected to the chambers. It is noted that both resistors may be any fluid resistor known in the art. In their most simplistic form, resistors are essentially narrow tubes or capillaries which are dimensioned so as to allow a maximum flow rate there through. Thus, regardless of the flow rate of fluid from either chamber, resistors act as restrictors and govern the maximum rate. Resistors are preferably connected to a collecting duct, which is in turn connected to a tube or capillary in communication with catheter.

In operation, a doctor and/or other medical professional may easily utilize the pump so as to provide different flow rates of medication to a patient. Initially, the pump may be implanted into the body of a patient by well-known methods for implanting such implantable devices.

Once the pump is implanted in the body of a patient, a medical professional may pick and choose which chambers to fill. Filling either chamber may provide either a first or second flow rate of fluid. Depending upon the particular conditions of the patient (e.g., the patient's current level of pain, pain type, and/or cause of pain), the medical professional may determine what chambers to fill and what compounds to use. As the pump is designed to house a limited amount of medication fluid, it must be refilled regularly. A doctor or nurse may utilize the regularly scheduled replenishment procedure as an opportunity to further monitor the patient and determine the proper flow rate for treating the patient's infirmity. Thus, if a doctor determines that the patient requires more medication fluid to be directed to the afflicted area, he/she may simply fill both chambers.

Further, a controlling mechanism is preferably provided for selectively applying power to the magnets which control the pump. Many different such mechanisms are well known and widely utilized to control implantable devices. For example, it is possible to utilize dedicated hard wired controllers, infrared controllers, or the like, which controllers could be used in accordance with the present invention to control various elements. U.S. Pat. No. 6,589,205 (“the '205 patent”), the disclosure of which is hereby incorporated by reference herein, teaches the use of a wireless external control. As discussed in the '205 patent, such a wireless control signal may be provided through modulation of an RF power signal that is inductively linked with the pump. The '205 patent cites and incorporates by reference U.S. Pat. No. 5,876,425, the disclosure of which is also hereby incorporated by reference herein, to teach one such use of forward telemetry or the exchange of information and programming instructions that can be used with the present invention to control the pump and the various aforementioned elements that are varied in order to affect the flow rate. However, it is noted that similar external controllers may also be utilized. Such controllers can send control signals wirelessly (such as by IR, RF or other frequencies) or can be wired to leads that are near or on the surface of the patient's skin for sending control signals. Furthermore, a pump in accordance with the present invention may include safeguards to prevent the inadvertent signaling or improper programming of the pump. For example, the present invention could utilize a secure preamble code or encrypted signals that will be checked by software or hardware used for controlling the pump or even dedicated only for security purposes. This preamble code would prevent the inadvertent actuation of magnets, from being caused by outside unrelated remote control devices or signals and by other similar pump controllers.

Preferably, an additional controller may be provided to prevent a patient from over utilizing the patient controlled actuation features. Preferably, such controller may include a digital timer (i.e. a clock) that must time out (after a pre-selected interval of time before the patient can actuate the magnets again). Other safety precautions may be used, such as passwords, hardware or software keys, encryption, multiple confirmation requests or sequences, etc. by the software or hardware used in the programming of the pump to prevent over-use of the patient controlled dose.

The electronics and control logic used with the present invention for control of the magnets may be located internally with or in the implantable pump and/or externally with any external programmer device used to transmit pump programming information to control the pump. The electronics can also be used to perform various tests, checks of status, and even store information about the operation of the pump or other physiological information sensed by various transducers.

An external programmer device may also be avoided by incorporating the necessary logic and electronics in or near or in the implantable pump such that control can be accomplished, for example, via control buttons or switches or the like that can be disposed on or below the surface of the skin. Of course, necessary precautions would need to be taken so that inadvertent changing of programming is again avoided.

The invention is illustrated further by the following examples that are not to be construed as limiting the invention in scope to the specific procedures described in it.

EXAMPLES

### Example 1

Intrathecal Infusion of Ziconotide and Morphine Using a Dual Chamber Programmable Implantable Pump in Patients Who do not Have Adequate Pain Relieve with Ziconotide

### Patients

Patients have severe chronic pain that is not adequately controlled by intrathecally delivered ziconotide. The old single chamber pump is removed and a new dual chamber programmable pump is implanted.

### Ziconotide

Ziconotide is supplied at a concentration of 25 μg/mL in 20 mL single-use vials and at a concentration of 100 μg/mL in 1 and 5 mL single-use vials. The first chamber of the new pump is rinsed and then filled with ziconotide according to instructions in the prescribing information. The pump is programmed so that the same daily ziconotide dose is delivered as with the old pump.

### Morphine

INFUMORPH® (preservative-free morphine sulfate sterile solution) is supplied in 20 mL single-use glass ampules at a morphine concentration of either 10 or 25 mg/mL. The second chamber of the new pump is rinsed and then filled with INFUMORPH® according to instructions in the prescribing information. The pump is programmed so that a proper dosage of INFUMORPH® is delivered to a patient. The recommended initial lumbar intrathecal dose range in patients with no tolerance to opioids is 0.2 to 1 mg/day. Doses for individuals who have some degree of opioid tolerance varies from 1 to 10 mg/day. The upper daily dosage limit for each patient must be individualized. It is possible that doses need to increase over time. Doses above 20 mg/day should be employed with caution since they may be associated with a higher likelihood of serious side effects.

### Example 2

Intrathecal Infusion of Ziconotide and Morphine Using a Dual Chamber Programmable Implantable Pump in Patients Who do not Have Adequate Pain Relieve with Intrathecal Morphine

### Patients

Patients have severe chronic pain that is not adequately controlled by intrathecally delivered morphine at doses of 2-20 mg/day. The old single chamber pump is removed and a new dual chamber programmable pump is implanted.

### Ziconotide

Ziconotide is supplied at a concentration of 25 μg/mL in 20 mL single-use vials and at a concentration of 100 μg/mL in 1 and 5 mL single-use vials. The first chamber of the new pump is rinsed and then filled with ziconotide according to instructions in the prescribing information. The pump is programmed to start delivering ziconotide at an initial dose of 1.2 μg/day and titrated with increases of not more than 0.5 μg/24 hours no more often than once weekly. The dose of IT ziconotide is adjusted according to the severity of pain, the patient's response to therapy, and the occurrence of adverse reactions. The maximum recommended dose is 19.2 μg /day (0.8 μg/hour).

### Morphine

INFUMORPH® (preservative-free morphine sulfate sterile solution) is supplied in 20 mL single-use glass ampules at a morphine concentration of either 10 or 25 mg/mL. The second chamber of the new pump is rinsed and then filled with INFUMORPH® according to instructions in the prescribing information. The pump is programmed to deliver the same daily morphine dose as with the old pump.

### Example 3

### Intrathecal Infusion of Ziconotide and Baclofen Using a Dual Chamber Programmable Implantable Pump in Patients Who Have Spasticity and Severe Chronic Pain for Whom Intrathecal Therapy is Warranted and Who are Intolerant to or Refractory to Other Treatment

### Patients

Patients have spasticity and severe chronic pain for whom intrathecal therapy is warranted and who are intolerant to or refractory to other treatment. A dual chamber programmable pump is implanted.

### Ziconotide

Ziconotide is supplied at a concentration of 25 μg/mL in 20 mL single-use vials and at a concentration of 100 μg/mL in 1 and 5 mL single-use vials. The first chamber of the new pump is rinsed and then filled with ziconotide according to instructions in the prescribing information. The pump is programmed to start delivering ziconotide at an initial dose of 1.2 μg/day and titrated with increases of not more than 0.5 μg/24 hours and no more often than once weekly.

The dose of IT ziconotide is adjusted according to the severity of pain, the patient's response to therapy, and the occurrence of adverse reactions. The maximum recommended dose is 19.2 gg/day (0.8 gg/hour).

### LIORESAL®

LIORESAL® INTRATHECAL (baclofen injection) is packaged in single use ampules containing 0.05 mg/1 mL (50 μg/mL), 10 mg/20 mL (500 μg/mL), 10 mg/5 mL (2000 μg/mL), or 40 mg/20 mL (2000 μg/mL). The second chamber of the new pump is rinsed and then filled with LIORESAL® according to instructions in the prescribing information. The pump is programmed as following: the screening dose of LIORESAL® that gives a positive effect is doubled and administered over a 24-hour period, unless the efficacy of the bolus dose is maintained for more than 8 hours, in which case the starting daily dose is the screening dose delivered over a 24-hour period. No dose increases is given in the first 24 hours (i.e., until a steady state is achieved).

For adult patients with spasticity of spinal cord origin, after the first 24 hours, the daily dosage should be increased slowly by 10-30% increments and only once every 24 hours, until the desired clinical effect is achieved.

For adult patients with spasticity of cerebral origin, after the first 24 hours, the daily dose should be increased slowly by 5-15% only once every 24 hours, until the desired clinical effect is achieved.

For pediatric patients, after the first 24 hours, the daily dose should be increased slowly by 5-15% only once every 24 hours, until the desired clinical effect is achieved.

All publications and patent applications cited in this specification are herein incorporated by reference as if each individual publication or patent application were specifically and individually indicated to be incorporated by reference. Although the foregoing has been described in some detail by way of illustration and example for purposes of clarity of understanding, it will be readily apparent to those skilled in the art in light of the teachings of the specification that certain changes and modifications may be made thereto without departing from the scope of the appended claims.

What is claimed is:

1. A method for reducing pain in a patient, comprising:
intrathecally administering to a patient in need thereof (a) a pharmaceutical formulation comprising an effective amount of an omega conopeptide and an anti-oxidant, and (b) an effective amount of one or more compounds selected from the group consisting of morphine, hydromorphone, fentanyl, sufentanil, bupivacaine, baclofen, clonidine, buprenorphine, and their pharmaceutically acceptable salts thereof,
wherein the pharmaceutical formulation and the one or more compounds are administered by a drug dispensing implantable pump having a chamber for the omega conopeptide and one or more chambers for the one or more compounds to ensure that the pharmaceutical formulation retains at least 80% of the ziconotide activity at 37° C. for at least 7 days.

2. The method according to claim 1, wherein the omega conopeptide is ziconotide.

3. The method according to claim 1, wherein (a) and (b) are administered concomitantly.

4. The method according to claim 1, wherein (a) and (b) are administered sequentially.

5. The method according to claim 1, wherein said pharmaceutical formulation retains at least 80% ziconotide activity at 37° C. for at least one month.

6. The method according to claim 1, wherein said compound is morphine.

7. The method according to claim 1, wherein said compound is hydromorphone.

8. The method according to claim 1, wherein said compound is baclofen.

9. The method according to claim 1, wherein said antioxidant is methionine.

10. The method according to claim 1, wherein said pharmaceutical formulation has pH between 4 and 4.5.

11. The methods claim 1, further comprising adjusting the flow rate of each chamber independently for independent dose adjustments of the omega conopeptide and the one or more compounds.

12. The methods claim 11, wherein the flow rate of each chamber is adjusted based on the patient's response to therapy and the incidence of adverse events.

13. The method of claim 1, wherein the patient is suffering from severe chronic pain.

14. The method of claim 1, wherein the patient is suffering from spasticity, severe chronic pain due to cancer, failed back syndrome, CRPS, neuropathic pain, nocieceptive pain and mixed neuropathic and nociceptive pain.

15. A method for reducing pain in a patient, comprising: administering to a patient in need thereof (a) a pharmaceutical formulation comprising an effective amount of an omega-conopeptide and an anti-oxidant, and (b) an effective amount of a compound selected from the group morphine, hydromorphone, baclofen, and their pharmaceutically acceptable salts thereof, wherein said administering is by intrathecal infusion using a drug dispensing implantable pump having a chamber for the omega-conopeptide and a separate chamber for the compound, so that the omega-conopeptide retains its stability in the pharmaceutical formulation.

16. The method according to claim 15, wherein the omega conopeptide is ziconotide.

17. The method according to claim 16, wherein said pharmaceutical formulation retains at least 80% ziconotide activity at 37° C. for at least one month.

18. The method according to claim 15, wherein said antioxidant is methionine.

19. The method according to claim 15, wherein said pharmaceutical formulation has a pH between 4 and 4.5.

20. The method according to claim 15, wherein (a) and (b) are administered concomitantly.

21. A method for reducing severe, chronic pain in a patient having an implantable pump, comprising:
placing ziconotide in a first chamber of a pump implanted in a patient having severe, chronic pain,
placing morphine in a second chamber of the pump, and
administering the ziconotide and the morphine so that the ziconotide retains at least 80% of the ziconotide activity at 37° C. for at least 7 days.

22. The method according to claim 21, wherein the administering is intrathecally administering.

23. The method according to claim 21, further comprising programming the pump to administer the ziconotide or the morphine each at a specified rate.
